(19) United States
(12) Patent Application Publication
(10) Pub. No.: US 2013/0110181 A1
(43) Pub. Date: May 2, 2013

(54) CRANIAL FIXATION DEVICE

(71) Applicant: Carmen Piotrowski, Schaffhausen (CH)

(72) Inventor: Carmen Piotrowski, Schaffhausen (CH)

(73) Assignee: PINA-MEDIZINTECHNIK-VERTRIEBS AG, Neuhausen am Rheinfall (CH)

(21) Appl. No.: 13/457,137

(22) Filed: Apr. 26, 2012

(30) Foreign Application Priority Data
Oct. 28, 2011 (EP) 11187209.9

(51) Int. Cl. A61B 17/84

(52) U.S. Cl. 606/324

(57) ABSTRACT

A cranial fixation device has a cranial clamp with first and second clamping elements. The first clamping element is fixedly coupled to a rod that extends from the first clamping element and ends in an end section. The second clamping element is movably coupled to the rod and is further coupled to a hand-operable applicator having a holding part, a pushing part, and a connecting part. The end section of the rod is fastened to the holding part, and the hand-operable applicator is configured such that pushing of the connecting part towards the rod increases the distance between the holding part and the pushing part to thereby move the second clamping element in the direction of the first clamping element. The rod has a cross section which at least partly has a vertex or vertices.

### BACKGROUND ART

Document EP 1 985 248 A1 discloses a cranial fixation device for the fixation of neighbouring cranial plates. All of the cranial plates taken together form the cranial flap, known in German as the Schädelkalotte. The published document discloses a hand-operated cranial fixation device comprising a cranial clamp, as well as an applicator, wherein the cranial clamp may be arranged on the cranial bones with the help of the applicator and thereafter can be tightened through operation of the applicator, to thereby fasten the cranial clamp to the cranial flap.

Insertion and fastening of the cranial clamp could be improved for this, in itself, well-proven cranial fixation device.

### PRESENTATION OF THE INVENTION

The problem to be solved by the invention is to provide a cranial fixation device which enables the cranial clamp to be arranged on and fastened to the cranial plates in a simple manner.

This problem is solved by a cranial fixation device having the features of claim 1. Dependent claims 2 to 13 relate to further advantageous embodiments of cranial fixation devices. The problem is also solved by a hand-operable applicator having the features of claim 14. The problem is also solved by a set comprising the hand-operable applicator and a plurality of cranial clamps.

In particular, the problem is solved by a cranial fixation device comprising a cranial clamp with a first and a second clamping element, as well as a rod, wherein the first clamping element is fixedly connected to the rod, and wherein the rod extends from the first clamping element and ends in an end section, and wherein the second clamping element is movably arranged on the rod, also comprising a hand-operable applicator with a holding part, a pushing part, as well as a connecting part, wherein the holding part and the pushing part are connected with each other via the connecting part, wherein the end section of the rod is fastened to the holding part, and wherein the pushing part is arranged in the direction of extension of the rod between the holding part and the second clamping element, and wherein the connecting part extends in the direction of extension of the rod and is distanced from the rod in such a way that a pushing of the connecting part towards the rod increases the distance between the holding part and the pushing part, in order to thereby move the second clamping element in the direction of the first clamping element through the effect of the pushing part acting upon it, wherein the rod has a cross-section which at least partly has a vertex or vertices.

The cranial fixation device according to the invention has the advantage that the rod has substantially more stable properties, which enables the first clamping element to be better arranged on the cranial plates. In a particularly advantageous embodiment, the cross-section of the rod has several vertices.

In a particularly advantageous embodiment, the second clamping element has a recess, through which the rod extends, wherein the second clamping element comprises a guiding part which borders the recess on one side thereby forming a guiding surface, wherein the second clamping element also has a ratcheting part respectively a self-locking part which is directed towards the recess and is arranged opposite to the guiding surface, wherein the rod has a structured surface on its side facing the self-locking part and has a first guiding side on its side facing the guiding surface, such that the rod, on the one hand, lies against the guiding surface via its first guiding side and, on the other hand, the self-locking part is functionally connected to the surface of the rod. This embodiment allows a particularly secure and reliable connection between the rod and the second clamping element. After the fastening of the cranial clamp to the cranial plates, the rod is cut off where it extends beyond the cranial clamp, for which reason it is of central importance that the first and second clamping elements of the cranial clamp, which is to remain on the skull, are connected with each other securely and with long-term reliability. The embodiment according to the invention has the further advantage that the second clamping element is mostly or even completely prevented from tipping over or twisting with respect to the rod. This allows the second clamping element to be moved along the rod in the direction of the first clamping element in a reproducible and exact manner.

The structured surface is preferably toothed, wherein the structured surface advantageously extends along a level surface, which enables a particularly advantageous engagement of the self-locking part in the structured surface, such that the second clamping element is held particularly well to the rod.

In a further advantageous embodiment the rod comprises two structured surfaces extending along the direction of extension of the rod, wherein the two structured surfaces are advantageously arranged at 90° to one another. The second clamping element advantageously comprises two self-locking parts, which are arranged such that one self-locking part engages with one of the two structured surfaces and the other with the other, such that the second clamping element is held particularly well to the rod.

In a particularly advantageous embodiment, the overall height of the second clamping element is high enough that the self-locking part is arranged so as to extend within this overall height. This embodiment has the advantage that the self-locking part is particularly well-protected during the cutting off of the rod, such that it is ensured that the self-locking part is not injured during and after the cutting off of the rod, and that the first and second clamping elements are securely and reliably connected with each other via the rod.

In a further advantageous embodiment, the holding part also comprises a self-locking part, which engages in the end section of the rod, in order to reliably attach the rod in the end section.

In a further advantageous embodiment, the holding part comprises a passage with a cross-section substantially corresponding to that of the rod, such that the rod cannot escape sideways at the passage. This enables, on the one hand, a particularly reliable holding of the rod in the end section. Also, the position of the rod is precisely defined with respect to the holding part and the applicator, which is connected to the holding part, which provides the advantage, that the first clamping element, which is at the end of the rod, can be positioned particularly precisely and also simply.

The applicator is formed such that operation of an operating section occurs at an angle to the direction of extension of the rod preferably essentially perpendicularly to the direction of extension of the rod. In a preferred embodiment, the cranial fixation device is also held at the operating section while the cranial clamp is being inserted into the cranial bones. This gives the advantage that the cranial fixation device can, using a single hand, be both positioned in the cranial bones and thereafter the cranial clamp can be tightened, i.e., fastened, through a pressing of the operating section. Due to the operating section being operated at an angle to the direction of extension of the rod, the advantage is produced that the cranial clamp may be positioned and fasten very pleasantly and precisely through the cranial clamp being positioned in a first operative step and the cranial clamp being fastened in a second operative step, wherein a force directed at an angle to the rod is exerted on the operating section during fastening, which results in it being possible to hold the cranial clamp relatively precisely in its position even during fastening, such that positioning and fastening of the cranial clamp can be carried out using a single hand. The cranial fixation device according to the invention thereby has the advantage that it can be fastened to the cranial flap in a manner that is secure, fast and very pleasant for a surgeon. After tightening, i.e., fastening, of the cranial clamp, all that remains, such that the cranial clamp is finally placed, is to part off the rod and the next cranial clamp can be set.

In a preferred embodiment, the cranial fixation device is made to be disposable through its comprising each of a hand-operable applicator and a cranial clamp connected thereto, wherein the hand-operable applicator is disposed of after positioning and fastening of the cranial clamp. This embodiment has the advantage that a surgeon can fasten a plurality of cranial clamps to the cranial flap in quick succession.

In a further embodiment, the hand-operable applicator can also be used multiple times, wherein the applicator has a fastening section to which one cranial clamp is fastenable at a time, for inserting it into the cranial flap and fastening it with the cranial flap. An advantage of this embodiment is to be seen in that a set may be provided to a surgeon comprising an applicator and a plurality of cranial clamps, even, if necessary, differently formed cranial clamps. A further advantage of this embodiment is to be seen in that cranial clamps of other manufacturers can also be inserted in the cranial flap and fastened with the cranial flap with the hand-operable applicator.

In the following, several embodiment examples are explained in detail.

### SHORT DESCRIPTION OF THE DRAWINGS

The drawings used to explain the embodiment examples show:

FIG. 1 a perspective view of a cranial fixation device;

FIG. 2 a plan view of the cranial fixation device of FIG. 1;

FIG. 3 a side view of the cranial fixation device of FIG. 1;

FIG. 4 a perspective view of an applicator;

FIG. 5 a side view of a cranial clamp, wherein one of the clamping elements is presented in cross-section;

FIG. 6 a fixation device of the applicator in detail;

FIG. 7 a view of the applicator along section A-A;

FIG. 8 a cranial fixation device which has been operated, such that the cranial clamp is drawn together;

FIG. 9 a plan view of a second clamping element;

FIG. 10 a cross-section through a rod;

FIG. 11 a detail view of a structured surface of a rod;

FIG. 12 a drawn-together cranial clamp in sideview, wherein the second element is presented in cross-section;

FIG. 13 the cranial clamp of FIG. 12 in perspective view;

FIG. 14 the cranial clamp of FIG. 12 after the parting off of the rod;

FIG. 15 a plan view of a further embodiment example of a second clamping element;

FIG. 16 a cross-section through a further embodiment example of a rod;

FIG. 17 a cross-section through a further embodiment example of a rod as well as schematically a second clamping element;

FIG. 18 cross-sections through two further embodiment examples of a rod.

In the drawings, the same parts have fundamentally been provided with the same reference numerals.

### WAYS OF CARRYING OUT THE INVENTION

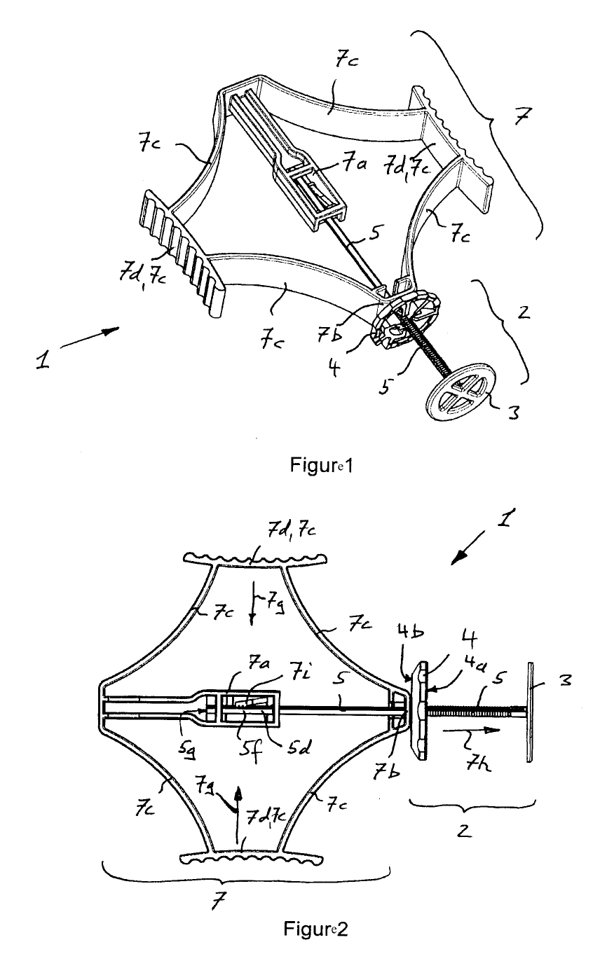

FIG. 1 shows a cranial fixation device 1 in a perspective view, consisting of a cranial clamp 2 and a hand-operable applicator 7. In a preferred embodiment, the applicator 7 and the cranial clamp 2 are formed as separate parts, which are connected with one another after their manufacture to form the cranial fixation device 1. The cranial clamp 2 comprises a first clamping element 3, a second clamping element 4 as well as a rod 5. The first clamping element 3 is connected in a fixed way to the rod 5, and the second clamping element 4 is mounted on the rod 5 so as to be movable in the direction of extension of the rod 5 to the first clamping element 3. The applicator 7 comprises a pushing part 7b as wells as a holding part 7a, wherein the pushing part 7b and the holding part 7a are connected with one another via the flexible connecting part 7c. In an advantageous embodiment, the parts of the connecting part 7c which run substantially parallel to the rod are formed as an operating section 7c, i.e., as a contact/resting surface for the finger. In a particularly advantageous embodiment, as shown in FIG. 1, the operating section 7d extends beyond the subsequent connecting part 7c on one or both sides, such that there is a particularly long operating section 7d in the direction of extension of the rod 5, which has the advantage that the contact/resting surface for the finger is particularly long and pleasant to hold. In a particularly advantageous embodiment, as shown in FIG. 1, the operating section 7d has a surface structure such as, for example, ribs, in order to be able to hold the operating section 7d well with fingers, and, in particular, with fingers over which a glove is drawn. Such an operating section 7d allows particularly good holding of the applicator 7, particularly goo insertion of the first clamping element 3 into the cranial bones and particularly good pressing together of the operating section 7d, in order to thereby fasten the cranial clamp 2.

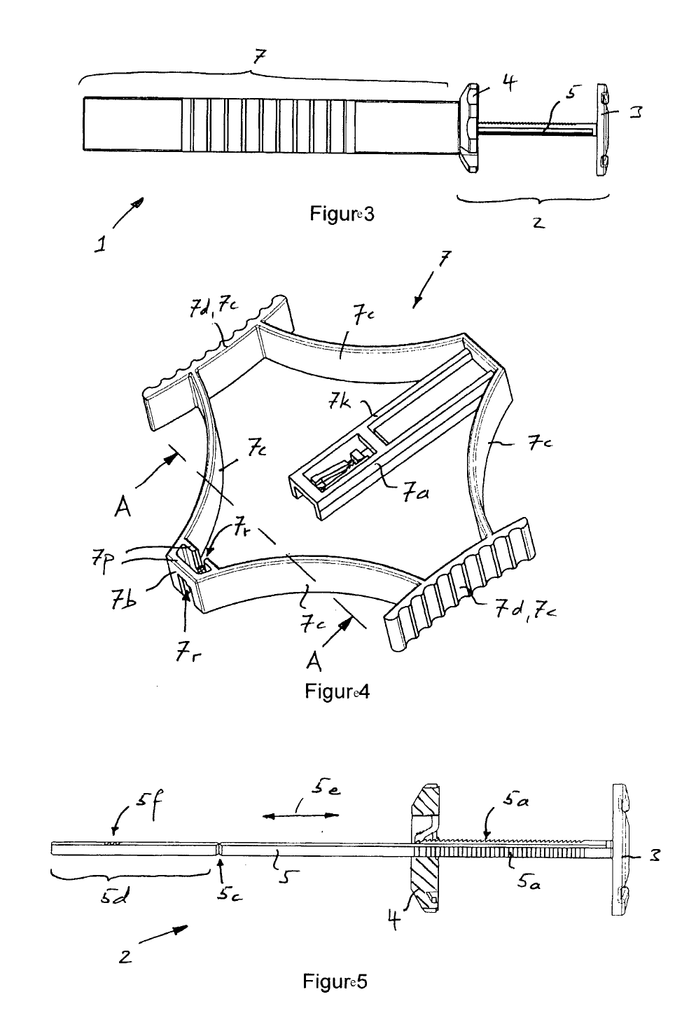

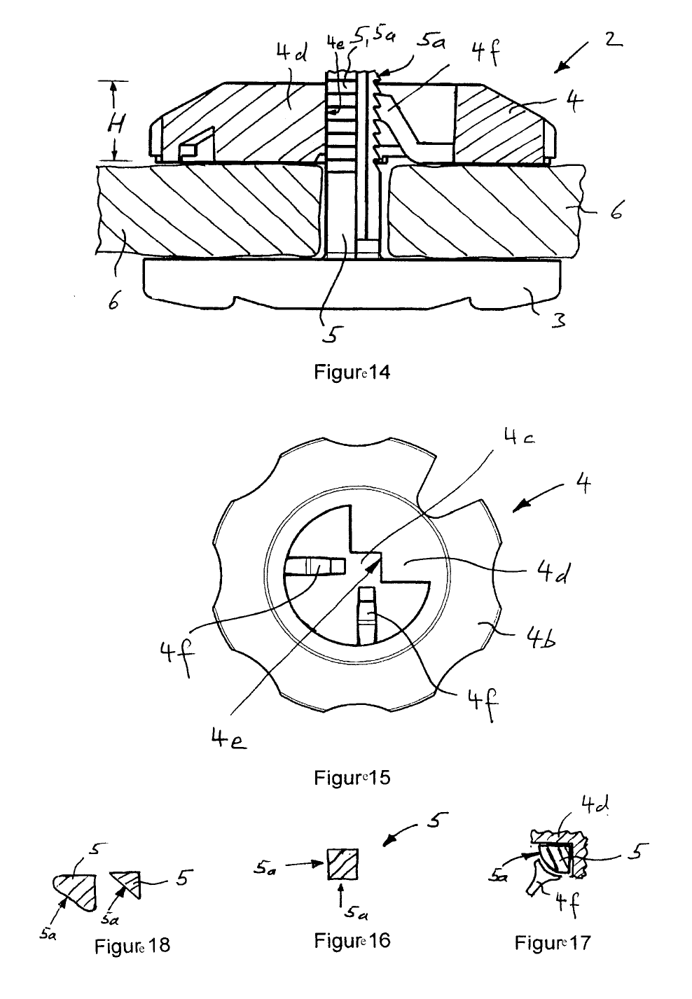

FIG. 2 shows a plan view of the cranial fixation device 1 of FIG. 1, and FIG. 3 shows a side view of the cranial fixation device 1. As may be seen from FIGS. 1 and 2, the cranial clamp 2 is arranged in the applicator 7 such that the rod 5 with an end section 5d is fixedly held in the holding part 7a. In a preferred embodiment, as shown in FIG. 2, the holding part 7a comprises a self-locking part 7i, which engages with the surface of the end section 5d and prevents a moving of the rod 5 with respect to the holding part 7a. The applicator 7 also comprises a pushing part 7b, which lies against the second clamping element 4. The flexible connecting part 7c of the applicator 7 is functional such that pressing together the two opposing operating sections 7d results in these being pressed together in direction of movement 7g which runs perpendicular to the direction of extension of the rod 5, which results in the pushing part 7b being moved in direction of movement 7h, which results in the second clamping part 4 being moved towards the first clamping part 3. The applicator 7 can be configured to extend in the most varied of ways to provide this functionality. FIG. 8 shows, for example, by dotted lines a further alternative extension of the flexible connecting parts 7c to provide this functionality. The connecting parts 7c could also be arranged on only one side, which is shown by the dotted line connecting parts 7c in FIG. 8, wherein the one finger would then lie against the operating section 7d and the other finger on the holding part 7a. The cranial fixation device 1 according to the invention is, for example, used, after a successful brain operation, to reattach a cranial plate, which was removed before the operation, to the cranial flap and to fasten it thereto. To do this, the cranial plate is laid against the cranial flap and the first clamping element is inserted between the cranial plate and the cranial flap. When doing this, the applicator 7 is preferably held with one finger on each of the operating sections 7d, such that the position of the first clamping element 3 can be guided by a corresponding hand movement and the first clamping element 3 can be precisely positioned on the cranial flap. The cranial fixation device 1 according to the invention, in particular, has the advantage that the first clamping element 3 is relatively stiffly connected with the applicator 7 via the rod 5, which results in being able to insert and position the first clamping element 3 very precisely with respect to the cranial flap and the cranial plate. In a second process step, the two operating sections 7d are pressed together in direction of movement 7g, which results in the second clamping element 4 being moved towards the first clamping element 3, such that cranial plates therebetween are fixed with respect to the cranial flap. In a subsequent step, the force exerted on the operating section 7d is reduced, such that the pushing part 7b is moved back, for example into the position shown in FIG. 2, whereupon the second clamping element 4 remains in the position previously taken up, as is shown in FIG. 8. In a subsequent step, as may be seen from FIG. 8, the rod 5 is cut preferably immediately after the second clamping element 4, so that the cranial clamp 2, as shown, for example, in FIG. 14, is fixed on the bone plates without or with preferably only a minimally sticking out rod 5.

FIG. 4 shows a perspective view of the applicator 7 without inserted cranial clamp 2. The applicator comprises a holding part 7a, which is connected with the flexible connecting part 7c via a holding arm 7k, wherein the flexible connecting part 7c is also connected with the pushing part 7c. In a preferred embodiment, the sections of the flexible connecting part 7c which run substantially parallel form the operating sections 7d. These preferably also stick out in the direction of extension of the rod 5, to enable pleasant holding and operating with the fingers. In an advantageous embodiment, the pushing part 7b also forms a guiding part 7p with a passage 7r, wherein, when a cranial clamp 2 is inserted, the rod 5 extends through the passage 7r and is guided sideways at least partially by the guiding part 7p. In FIG. 4 the passage 7r is U-formed. However, the passage could also be formed as a perforation, for example as a hole. In an advantageous embodiment, a further guiding part 7p with perforation 7r is arranged after the pushing part 7b, wherein both guiding parts 7p are advantageously arranged, as shown, to mirror each other such that the opening of the one passage 7r faces in one direction and the opening of the other passage 7r faces in the opposite direction. A rod 5 passing through both these passages 7 is guided and held particularly well by the guiding parts 7p, which results in the movement of the rod 5 being particularly well coupled to the movement of the operating sections 7d.

FIG. 5 shows a side view of the cranial clamp 2. In the depicted embodiment example, the surfaces of the rod 5 has toothing 5a in the area between the second clamping element 4 and the first clamping element 3. The rod 5 also comprises an end section 5d, i.e., a fastening section with a toothing 5f, at the end lying opposite the first clamping element 3. The rod can also, in one advantageous embodiment, comprise a thin portion 5c to produce better freedom of motion for the fastening section 5d with respect to the first clamping element 3.

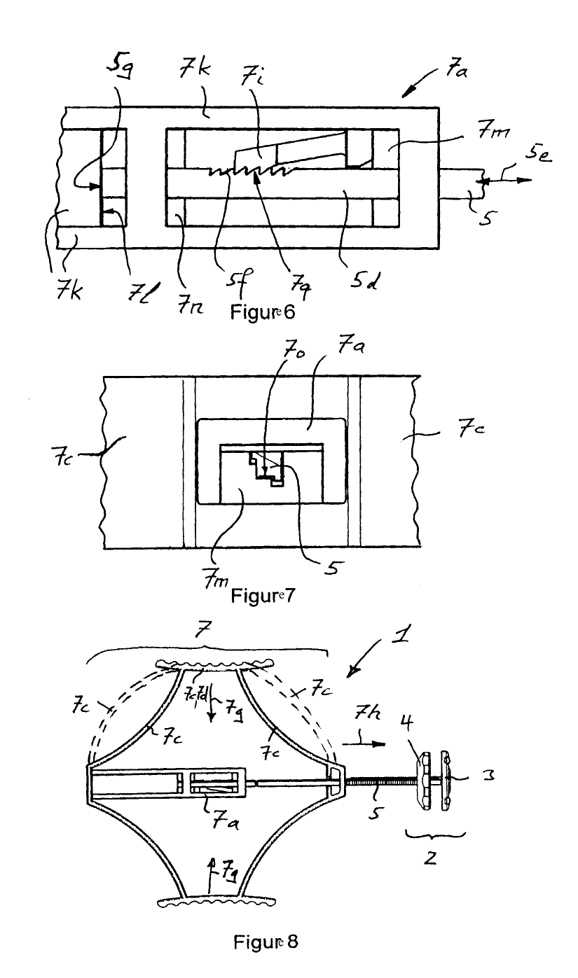

FIG. 6 shows an embodiment example of a holding part 7a of the applicator 7. In this embodiment example, the rod 5, as in FIG. 7, has a cross-section shown as a cross-section with multiple vertices. The rod 5 comprises an end section 5d with a toothing 5f. The holding part 7a comprises a self-locking part 7i with a toothing on its frontal end 7q, which are formed to mirror the toothing 5f. The self-locking part 7i is also elastically mounted perpendicular to the direction of extension 5e of the rod 5 and provides a prestressed force directed towards the rod 5. After introduction of the rod 5, this results in the end section 5d of the rod 5 being firmly mounted in the holding part 7a with respect to direction of movement 5e. In a further preferred embodiment the holding arm 7k also forms a pushing surface 7l, which the frontal end 5g of the end section 5d of the rod 5 lies against. This provides the advantage that a pressing force can also be exerted via the applicator 7 on the rod 5, respectively on the first clamping element 3.

FIG. 7 shows a cross-section view of the applicator 7 along line A-A shown in FIG. 4. In this embodiment example the holding part 7a comprises a guiding part 7m with a recess or, as the case may be, a passage 7o. The passage 7o is preferably formed to match the cross-section of the rod in such a way that the rod 5 is guided at its sides through the passage 7o and that no or only a minimal gap arises between the guiding part 7m and the rod 5, such that the rod 5 is guided at its sides by the guiding part 7m. This embodiment has the advantage that the rod 5 is held in the holding part particularly well and precisely, since, on the one hand, the frontal end 5g of the end section 5d of the rod 5 lies against the pushing surface 7l and, on the other hand, the rod 5 is held at its sides to the guiding part 7m and the rod 5 is firmly held in direction of movement 5e by the self-locking part 7i. In a preferred embodiment, as shown in FIG. 6, a further guiding part 7n is also arranged in the holding part 7a, which, for example, can be identical to the guiding part 7n shown on the right and also in FIG. 7, in order to additionally guide the rod 5 in the holding part 7a, such that the end section 5d cannot escape despite the forces exerted via the self-locking part 7i or despite the forces acting in direction 5e. This effects a particularly reliable connection of the self-locking part 7i with the engagement portion 5f of the rod 5.

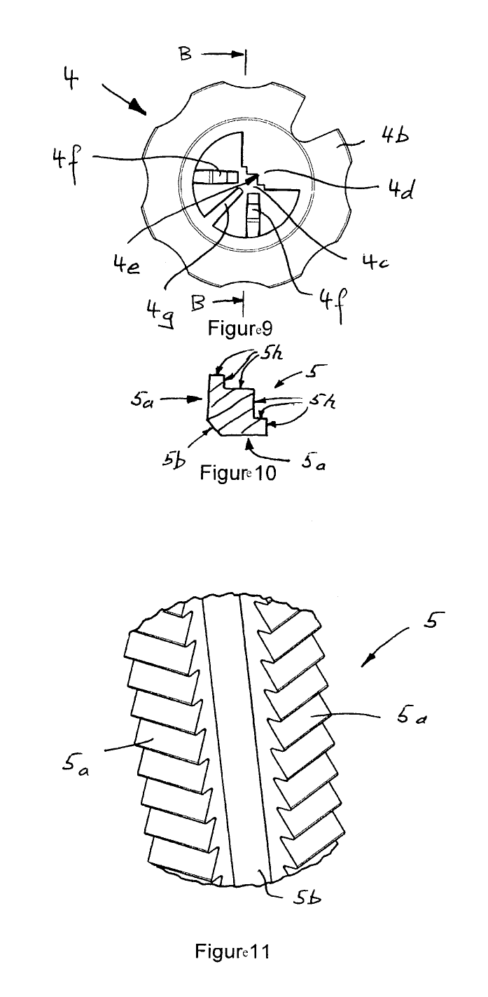

FIG. 9 shows an embodiment of a second clamping element 4 in plan view, wherein the outer surface 4b is shown, which is also indicated in FIG. 2. The second clamping element 4 comprises an opening 4c and comprises a guiding part 4d, which forms a guiding surface 4e extending perpendicularly to the plane of observation. Two self-locking parts 4f, in the form of latches, are arranged opposing this guiding surface 4e. In the embodiment example shown, there is also a guiding nib 4g protruding towards the opening 4c.

FIG. 10 shows the cross-section of a rod 5 which is intended to be accommodated by the second clamping element 4 shown in FIG. 9. The rod 5 comprises two first guiding sides 5h, which both lie against the guiding surface 4e of the second clamping element 4 where they pass through the second element 4. The rod 5 also comprises two faces with structured surfaces 5a, which are toothed and which extend perpendicular to one another in the embodiment example shown.

The rod 5 also comprises a second guiding side 5b, against which the guiding nib 4g preferably lies as soon as the rod 5 is inserted into the second clamping element 4. The guiding nib 4g advantageously results in the rod 5 being pressed against the guiding surface 4e and/or in the second clamping element 4 being arranged precisely and reproducibly with respect to the opening 4c or, as the case may be, with respect to the rod 5. In a particularly advantageous embodiment, the second clamping element 4 has a height H, as shown in FIG. 14, which can, for example be in the range of 1 to 5 mm, which results in the guiding surface 4e being as long as height H, which results in being able to reduce or completely prevent the second clamping element 4 tipping over with respect to the rod 5, in particular since the rod 5 preferably lies against the guiding surface 4e due to the guiding nib 4g acting on the rod 5 and/or to the self-locking parts 4f acting on the rod 5.

FIG. 11 shows a detailed perspective view of the rod 5 shown in FIG. 10 with a second guiding side 5b and two structured surfaces 5a in the form of toothing.

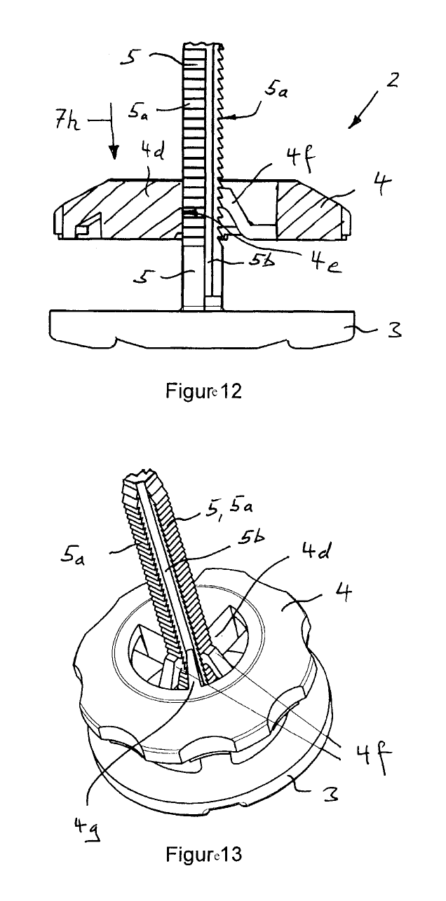

FIG. 12 shows a side view of the cranial clamp 2, wherein the second clamping element 4 is shown in cross-section, wherein the cross-section of the clamping element 4 is along line B-B shown in FIG. 9. The rod 5 is as shown in FIGS. 10 and 11 and the second clamping element 4 is as shown in FIG. 9 and has a latch 4f, the frontal end of which engages with the toothing 5a, such that it is possible to move the second clamping element 4 in the direction of motion 7h towards the first clamping element 3 and it is not possible to move it in the direction opposite to direction 7h. The embodiment example shown of the second clamping element 4 with guiding part 4d, guiding surface 4e and latch 4f has the advantage that motion of the second clamping element 4 in the opposite direction to direction 7h is not possible or is only possible through applying extraordinarily high forces which could break the latch 4f. This ensures that a cranial clamp 2 which has been fixated, i.e., tightened, holds together reliably and for the long term and does not fall apart.

FIG. 13 shows a perspective view of the arrangement shown in FIG. 12. In FIG. 13 it may be particularly clearly seen how the two self-locking parts 4f, which take the form of latches, engage with the toothings 5a and how the guiding nib 4g affects the second guiding side 5b to produce a reproducible and secure sliding of the second clamping element 4 along the rod 5 and to ensure a secure fastening of the second clamping element 4 with respect to the rod 5.

FIG. 14 shows a fixate cranial clamp 2, which is lying against bone plates and holds these together. In contrast to the arrangement shown in FIG. 12, in the arrangement of FIG. 14 the rod 5 is cut immediately after the second clamping element 4. In a particularly advantageous embodiment, the height H of the second clamping element 4 and the self-locking part 4f, in the form of a latch, are matched with respect to one another such that the self-locking part 4f extends completely inside the second clamping element 4, i.e., along height H inside the second clamping element 4. This embodiment has the advantage that, during the parting-off of the rod 5, for example with a pair of scissors or a pair of pliers, the self-locking part 4f is protected from being mechanically affected by the scissors or pliers, such that the self-locking part 4f cannot be damaged and thereby ensuring a secure connection between the second clamping element 4 and the rod 5 during and in particular after successful cutting.

FIG. 15 shows a plan view of a further embodiment example of a second clamping element 4, which, in contrast to the embodiment example shown in FIG. 9, does not have a guiding nib 4g and has a rectangular guiding surface 4e.

FIG. 16 shows a cross-section through a rod 5 which would be suitable for the second clamping element shown in FIG. 15. The rod shown in FIG. 16 has structured surfaces 5a on two sides, in the form of toothings.

FIG. 17 shows a further embodiment example of a cross-section of a rod 5 with a cross-section which at least partly has a vertex or vertices. The cross-section has three vertices. One side extends circularly or in a curved form and has a structured surface 5a which may, for example, be toothed as shown in FIG. 11, though curved.

The second clamping element 4 is only shown in part and comprises a guiding part 4d with a guiding surface 4e and a curved self-locking part 4f. This embodiment example also has the property that the rod 5 is pressed against the guiding surface 4e of the second clamping element 4 and the impinging self-locking part 4f is thereby securely held. The cross-section of the rod 5, as shown in FIG. 17, could be the same over the entire length of the rod 5, in which case the holding part 7a would have to be matched to this cross-section such that the end section 5d is securely held in the holding part 7a. The form of the cross-section of the rod 5 could also, however, change in its direction of extension, and, for example, be a rectangular cross-section left of the thin portion 5c, as shown in FIG. 5. In this way, for example, the embodiments of the holding part 7a shown in FIGS. 4, 6 and 7 would be suitable for holding the rod 5.

On the left of FIG. 18, a further embodiment example of a rod 5 is shown with a cross-section which at least partly has a vertex or vertices. The cross-section has one vertex. On the right of FIG. 18, a further embodiment example of a rod 5 is shown which has vertices throughout. The cross-section has three vertices. Both of the posts shown in FIG. 18 could be arranged, for example, in the second clamping element 4 shown schematically in FIG. 17, wherein the form of the self-locking part 4f would have to be matched to the form of the structured surface 5a. In the preferred embodiment, however, the cross-section of the rod 5 has vertices throughout, as shown in FIGS. 7, 10 and 16 as well as on the right in FIG. 18. It is particularly advantageous for the rod 5 to have the cross-section shown in FIG. 10 with a plurality of guiding sides 5h, which enables a particularly precise guiding of the rod 5 with respect to the second clamping element.

1. A cranial fixation device (1) comprising:
a cranial clamp (2) with a first and a second clamping element (3, 4), and a rod (5), wherein the first clamping element (3) is fixedly connected to the rod (5), and wherein the rod (5) is configured to extend from the first clamping element (3) and to end in an end section (5d), and wherein the second clamping element (4) is movably arranged on the rod (5);
a hand-operable applicator (7) with a holding part (7a), a pushing part (7b), and a connecting part (7c), wherein the holding part (7a) and the pushing part (7b) are connected with each other via the connecting part (7c),
wherein the end section (5d) of the rod (5) is fastened to the holding part (7a), and wherein the pushing part (7b) is arranged in the direction of an extension (5e) of the rod (5) between the holding part (7a) and the second clamping element (4), and
wherein the connecting part (7c) extends in the direction of the extension (5e) of the rod (5) and is distanced from the rod (5) in such as to allow pushing of the connecting part (7c) towards the rod (5) to increase a distance between the holding part (7a) and the pushing part (7b) to thereby move the second clamping element (4) in the direction of the first clamping element (3), and
wherein the rod (5) has a cross-section which at least partly has a vertex or vertices.

2. Device according to claim 1, wherein the second clamping element (4) has a recess (4c), through which the rod (5) extends, wherein the second clamping element (4) comprises a guiding part (4d) which borders the recess (4c) on one side thereby forming a guiding surface (4e), wherein the second clamping element (4) has a self-locking part (4f) which is directed towards the recess (4c) and is arranged opposite to the guiding surface (4e), and wherein the rod (5) has a structured surface (5a) on its side facing the self-locking part (4f) and has a first guiding side (5h) on its side facing the guiding surface (4e), such that the rod (5), on the one hand, lies against the guiding surface (4e) via its first guiding side (5h) and, on the other hand, the self-locking part ( 4f) is functionally connected to the surface (5a) of the rod (5).

3. Device according to claim 2, wherein the structured surface (5a) has a plurality of successive teeth in the direction of extension (5e) of the rod (5).

4. Device according to claim 3, wherein the toothed structured surface (5a) extends along a level surface.

5. Device according to claim 3 or 4, wherein the rod (5) has two toothed structured surfaces (5a), wherein the two structured surfaces (5a) are arranged at 90 degrees to one another.

6. Device according to claim 2 wherein, between the two structured surfaces (5a), the rod (5) has a second guiding side (5b) extending in the direction of extension (5e) of the rod (5).

7. Device according to claim 6, wherein the second clamping element (4) has a guiding nib (4g), which is arranged such that it faces the second guiding surface (5b) and preferably lies against it.

8. Device according to claim 2, wherein in the direction of extension (5e) of the rod (5), the second clamping element (4) has an overall height (H), and that the self-locking part (4f) extends within this overall height (H), and engages in the structured surface (5a) of the rod (5) within this overall height (H).

9. Device according to claim 1 wherein the end section (5d) of the rod (5) has a self-locking section (5f), and that the holding part (7a) has a self-locking part (7i) facing the self-locking section (5f), which engages in the self-locking section (5f) such that the rod (5) is fixed with respect to the holding part (7a).

10. Device according to claim 9, wherein the self-locking section (5f) has a plurality of successive teeth in the direction of extension (5e) of the rod (5), and that the self-locking part (7i) is sprung at an angle to the direction of extension (5e) and has a frontal end (7q) to engage in the teeth.

11. Device according to claim 9 or 10, wherein the holding part (7a) comprises a guiding part (7m) with a passage (7o) on the side facing the pushing part (7b), wherein the passage is formed such that the rod (5) passes through the passage (7o), and wherein the passage (7o) is at least partially formed to match the extension of the cross-section of the rod (5), such that the rod (5) can at least partially lie against the passage (7o).

12. Device according to claim 9 wherein the holding part (7a) is connected with the flexible connecting part (7c) via a holding arm (7k), and that the holding arm (7k) comprises a pushing surface (7l) which faces towards the rod (5) and which the frontal end (5g) of the end section (5d) of the rod (5) can lie against.

13. Device according to claim 1 wherein the applicator (7) is configured as a single-piece.

14. Hand-operable applicator (7) and/or cranial clamp (2) for a cranial fixation device (1) according to claim 1.

15. A kit comprising a hand-operable applicator (7) according to claim 14 and a plurality of cranial clamps (2) which are connectable with the holding part (7a) of the applicator (7).
